(19) United States
(12) Patent Application Publication
(10) Pub. No.: US 2019/0000773 A1
(43) Pub. Date: Jan. 3, 2019

(54) NON-INGESTIBLE TRANSDERMAL TAPE

(71) Applicant: Patrick Mitchell Eaves, Las Vegas, NV (US)

(72) Inventor: Patrick Mitchell Eaves, Las Vegas, NV (US)

(21) Appl. No.: 16/017,822

(22) Filed: Jun. 25, 2018

Related U.S. Application Data
(60) Provisional application No. 62/526,036, filed on Jun. 28, 2017.

(51) Int. Cl. A61K 9/70; A61K 9/00

(52) U.S. Cl. CPC A61K 9/7038; A61K 8/553; A61K 9/0014

(57) ABSTRACT

A non-ingestible transdermal sports tape has an occlusive nonwoven pre-cut tape and night recovery therapy formulation, or a focus therapy formulation or a pre-workout therapy formulation. The occlusive nonwoven pre-cut tape includes a dosage amount pad positioned on one side of the occlusive nonwoven precut tape, an adhesive seal material disposed on a surface of the occlusive non-woven pre-cut tape, a liner material covering the dosage amount pad and the adhesive seal material. The adhesive seal material is configured to adhere to epidermis of an animal body preventing evaporation of water from epidermis while providing a removeable seal. Ingredients of the therapy formulations include non-fermented panax ginseng content, schizandra vitamin B₆, vitamin B₁₂, and provides a transdermal absorption from one to eight hours per animal body.

### CROSS REFERENCE TO RELATED APPLICATIONS

The present application claims priority to and the benefit of co-pending U.S. Provisional Patent Application Ser. No. 62/526,036 filed on Jun. 28, 2017, entitled “NON-INGESTIBLE TRANSIDERMAL TAPE” (our reference 3041.001). This reference is hereby incorporated in its entirety.

### FIELD

The present embodiment generally relates to a non-ingestible transdermal sports tape.

### BACKGROUND

A need exists for transdermal dosage amounts of non-toxic ingredients to perform athletic performance, dexterity, focus, precision and accuracy in performance.

A need exists for various transdermal formulations infused in a patch to provide improved rest for athletes.

The present embodiments meet these needs.

### BRIEF DESCRIPTION OF THE DRAWINGS

The detailed description will be better understood in conjunction with the accompanying drawings as follows:

FIGS. 1A, 1B, and 1C depict cross sections of embodiments of the transdermal patch according to one or more embodiments.

FIG. 2 depicts a transdermal patch with a therapy formulation installed on an animal body one or more embodiments.

FIG. 3 shows a table of night recovery therapy formulations according to one or more embodiments.

FIG. 4 shows a table of pre-work out therapy formulations according to one or more embodiments.

FIG. 5 shows a table of focus therapy formulations according to one or more embodiments.

The present embodiments are detailed below with reference to the listed Figures.

### DETAILED DESCRIPTION OF THE EMBODIMENTS

Before explaining the present patches in detail, it is to be understood that the patches are not limited to the particular embodiments and that it can be practiced or carried out in various ways.

The present embodiments relate to a non-ingestible transdermal sports tape.

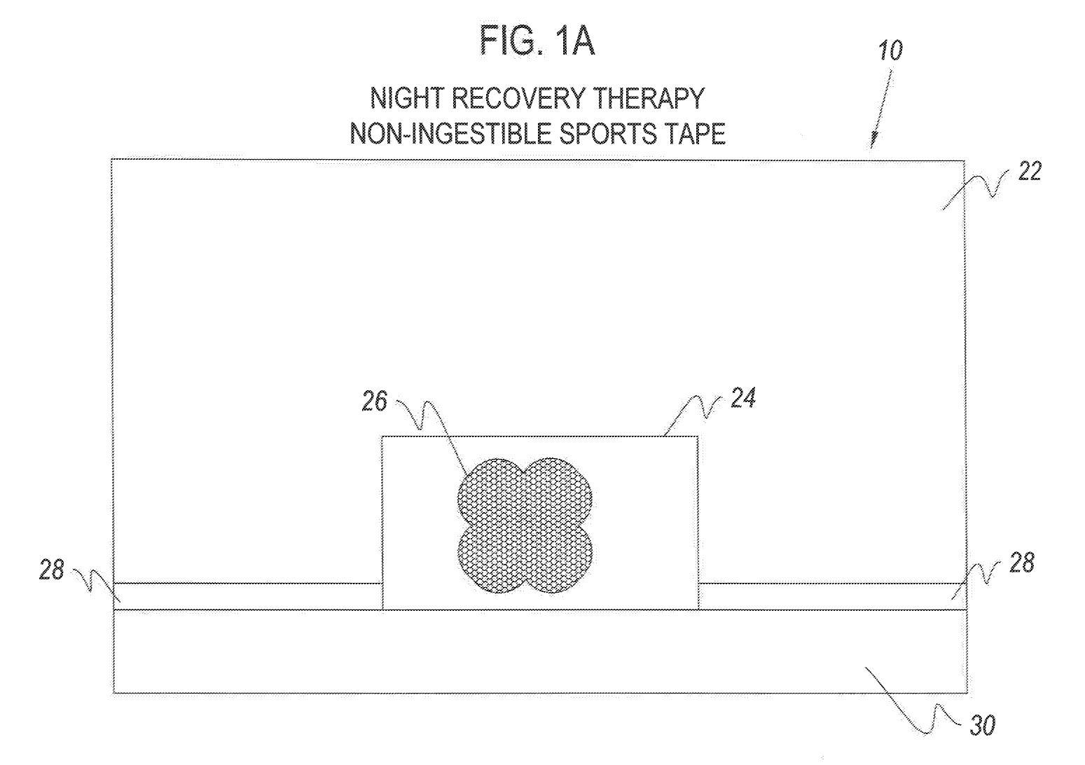

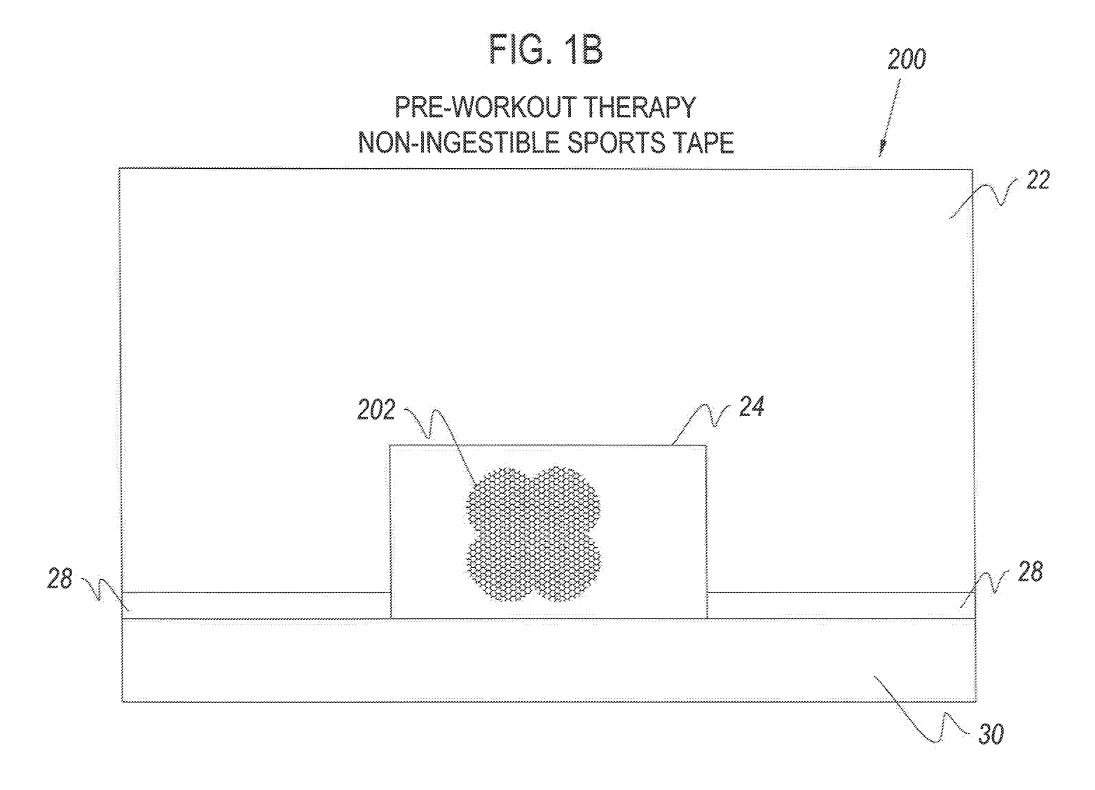

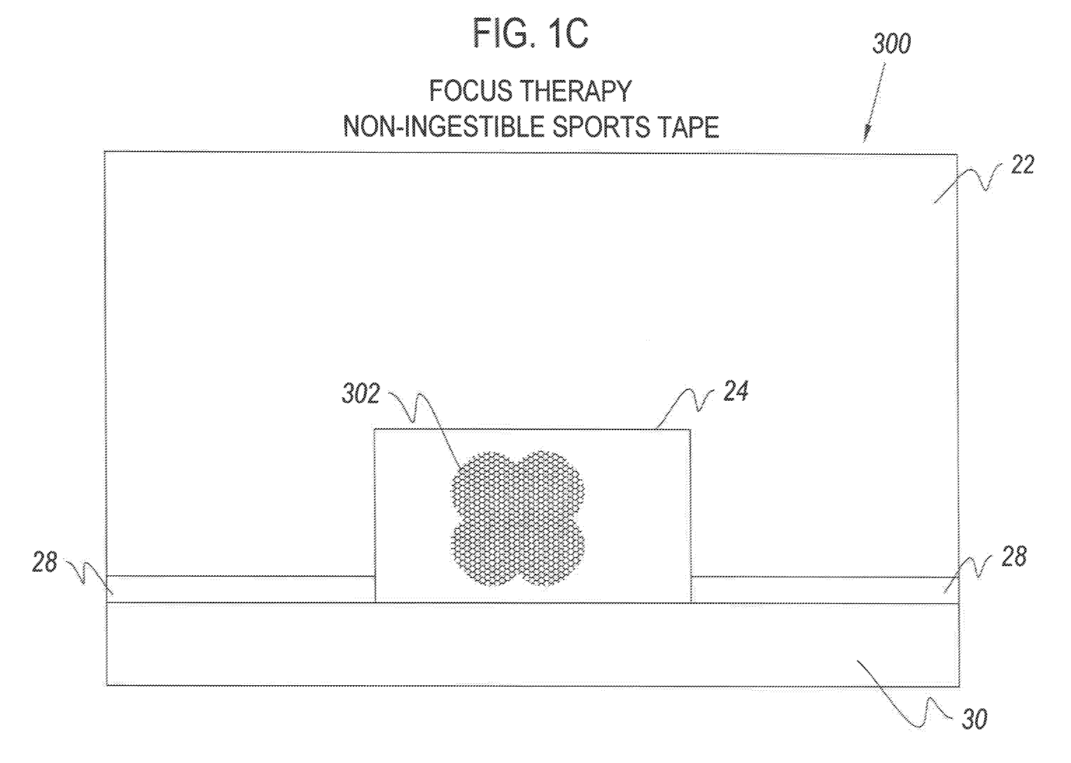

Turning now to the Figures, FIGS. 1A-1C depict cross sections of embodiments of the transdermal patch according to one or more embodiments.

The night recovery therapy non-ingestible transdermal sports tape 10 shown in FIG. 1A, the pre work out therapy non-ingestible transdermal sports tape 200 shown in FIG. 1B and the focus therapy non-ingestible transdermal sports tape 300 are each shown having an occlusive nonwoven pre-cut tape 22. In each embodiment, the occlusive nonwoven pre-cut tape is resistant to tearing and is flexible and elastic.

In embodiments, a dosage amount pad 24 is positioned on one side of the nonwoven precut tape, The dosage amount pad supports a dosage amount of a night recovery therapy formulation 26 as shown in FIG. 1A, a pre work out therapy formulation 202 as shown in FIG. 1B, and a focus therapy formulation 303 as shown in FIG. 1C.

An adhesive seal material 28 is disposed on the non-woven pre-cut tape surrounding and covering the dosage amount pad 24.

In embodiment, a liner material 30 covers the dosage amount pad 24 with the adhesive seal material 28.

The liner material 30 is configured to protect the dosage amount pad 24 and adhesive seal material from drying until the animal body 28 is ready to apply the night recovery therapy non-ingestible transdermal sports tape 10, the prework out non-ingestible transdermal sports tape 200 or focus non ingestible transdermal sports tape to the animal body.

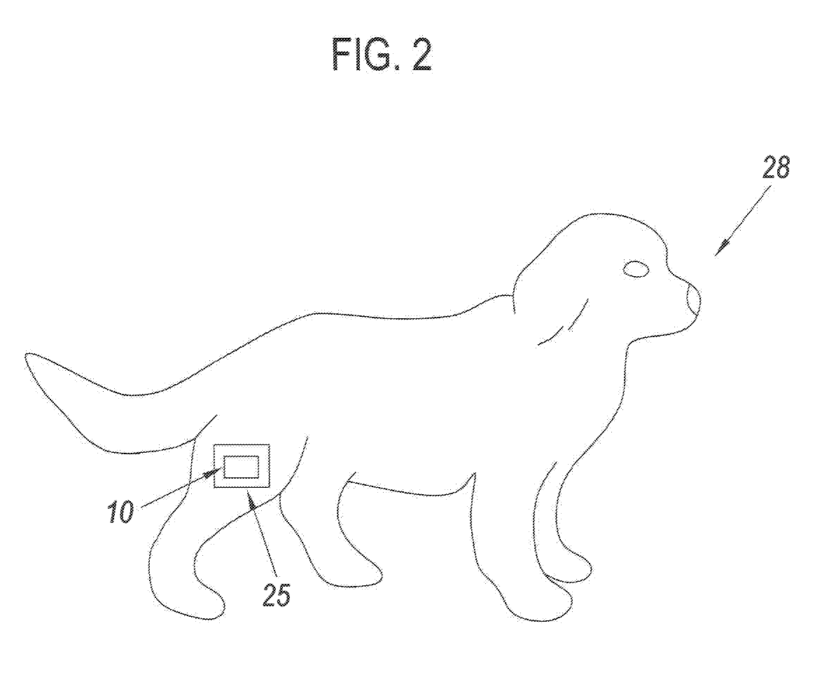

FIG. 2 depicts a transdermal patch with a therapy formulation installed on an animal body 28 with an epidermis 25.

The adhesive seal material is configured to adhere the non-woven pre-cut tape to an animal body 28 with an epidermis 25, providing a removable seal while preventing evaporation of water from epidermis 26 of the animal body 28.

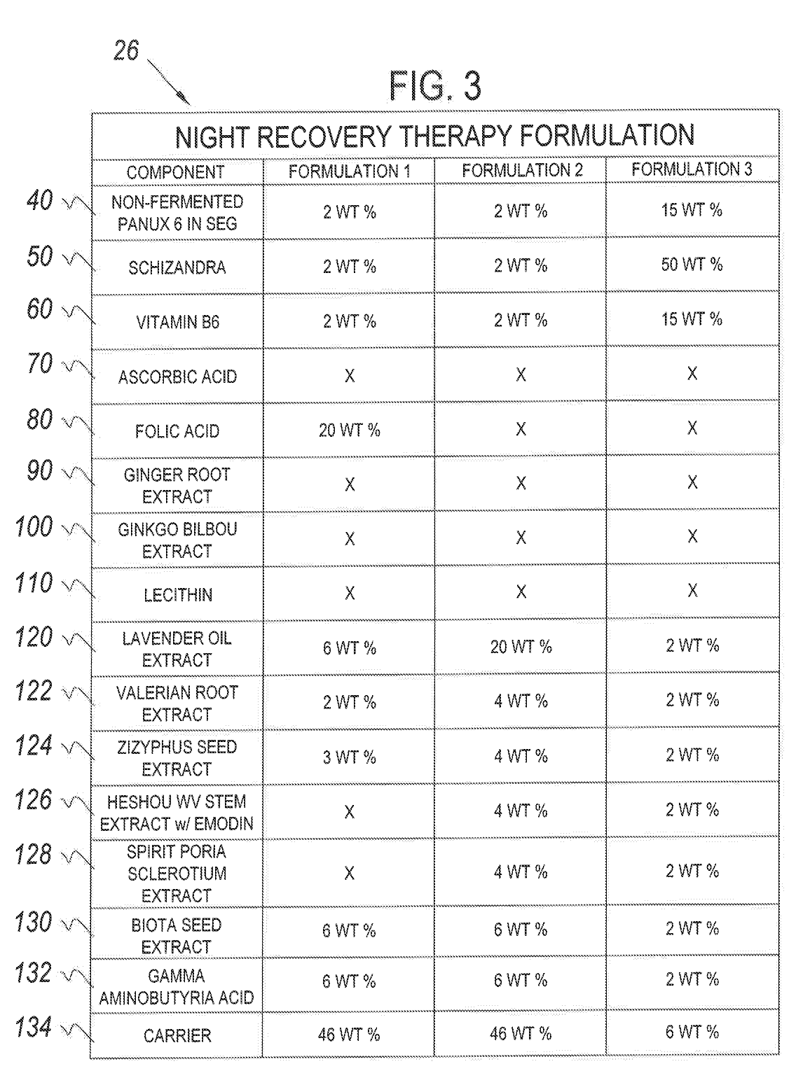

FIG. 3 shows a table of night recovery therapy formulations according to one or more embodiments.

The dosage amount of the night recovery therapy formulation 26 has a non-fermented panax ginseng 40 having a non-fermented panax ginseng weight percent content from 2 weight percent to 15 weight percent based on the total night recovery therapy formulation 26, a schizandra 50 having a schizandra weight percent content from 2 weight percent to 50 weight percent based on the total night recovery therapy formulation 26, and a vitamin B₆ 60 having a vitamin B₆ weight percent content from 2 weight percent to 15 weight percent based on the total night recovery therapy formulation 26; and wherein the epidermis temperature of an animal body with an epidermis 25 varying from 90 degrees to 98 degrees Fahrenheit liquefies the dosage amount of the night recovery therapy formulation 26 allowing transdermal absorption by the animal body for from 1 to 72 hours.

More specifically, Formulation 1 includes 2 weight percent of a non-fermented panax ginseng 40 based on the total night recovery therapy formulation 26, 2 weight percent of a schizandra 50 based on the total night recovery therapy formulation 26, and 2 weight percent of a vitamin B₆ 60 based on the total night recovery therapy formulation 26.

Formulation 1:

Formulation 1 includes 0 weight percent of Ascorbic Acid 70, 20 weight percent of Folic Acid 80, 0 weight percent of Ginger Root Extract 90, 0 weight percent of Ginkgo Biloba Extract 100, and 0 weight percent of Lecithin 110.

Formulation 1 includes 6 weight percent of lavender oil extract 120, 2 weight percent valerian root extract 122, 3 weight percent zizyphus seed extract 124, 0 weight percent He Shou Wu stem extract with Emodin 126, 0 weight percent Spirit Poria Selerotium Extract 128, 6 weight percent Biota Seed Extract 130, and 6 weight percent Gamma Aminobutyric Acid 132.

46 weight percent of a carrier, such as glycerin is also depicted.

The formulation components are mixed together in one pot then deposited on the dosage amount pad.

Formulation 2:

Formulation 2 is includes 2 weight percent of a non-fermented panax ginseng 40 based on the total night recovery therapy formulation 26; 2 weight percent of a schizandra 50 based on the total night recovery therapy formulation 26; and 2 weight percent of a vitamin B₆ 60 based on the total night recovery therapy formulation 26.

In embodiments, Formulation 2 includes 0 weight percent of Ascorbic Acid 70, 0 weight percent of Folic Acid 80, 0 weight percent of Ginger Root Extract 90, 0 weight percent of Ginkgo Biloba Extract 100, and 0 weight percent of Lecithin 110.

Additionally, Formulation 2 includes 20 weight percent of lavender oil extract 120, 4 weight percent valerian root extract 122, 4 weight percent zizyphus seed extract 124, 4 weight percent He Shou Wu stem extract with Emodin 126, 4 weight percent Spirit Poria Sclerotium Extract 128, 6 weight percent Biota Seed Extract 130, and 6 weight percent Gamma Aminobutyric Acid 132.

46 weight percent of a carrier, such as glycerin is also depicted.

The formulation components are mixed together in one pot then deposited on the dosage amount pad.

Formulation 3:

Formulation 3 is includes 15 weight percent of a non-fermented panax ginseng 40 based on the total night recovery therapy formulation 26, 50 weight percent of a schizandra 50 based on the total night recovery therapy formulation 26, and 15 weight percent of a vitamin B₆ 60 based on the total night recovery therapy formulation 26.

In embodiments, Formulation 3 includes 0 weight percent of Ascorbic Acid 70, 0 weight percent of Folic Acid 80, 0 weight percent of Ginger Root Extract 90, 0 weight percent of Ginkgo Biloba Extract 100, and 0 weight percent of Lecithin 110.

In addition, Formulation 3 includes 2 weight percent of lavender oil extract 120, 2 weight percent valerian root extract 122, 2 weight percent zizyphus seed extract 124, 2 weight percent He Shou Wu stem extract with Emodin 126, 2 weight percent Spirit Poria Sclerotium Extract 128, 2 weight percent Biota Seed Extract 130, and 2 weight percent Gamma Aminobutyric Acid 132.

6 weight percent of a carrier, such as lecithin and isopropyl palmitate is also depicted.

The formulation components are mixed together in one pot then deposited on the dosage amount pad.

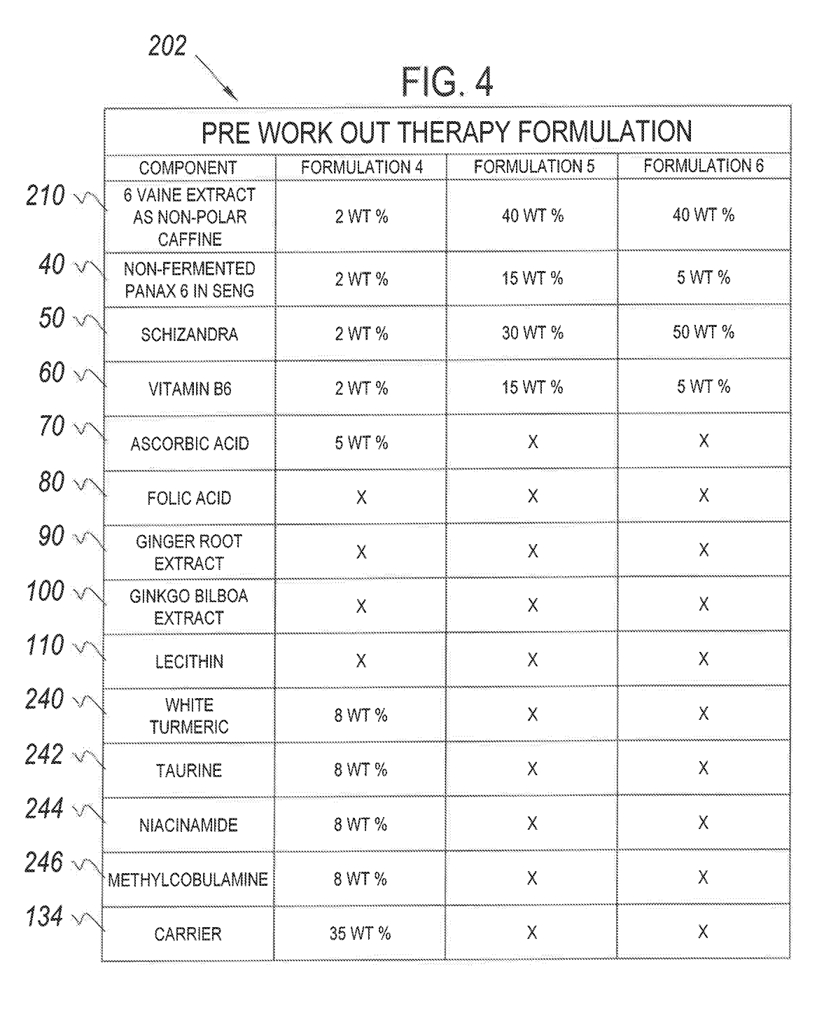

FIG. 4 shows a table of pre-work out therapy formulations usable herein.

The dosage amount of the pre-work out therapy formulation 202 includes (i) a guaranine extract as a caffeine 210 with a guanine extract weight percent content from 2 weight percent to 40 weight percent based on the total pre-work out therapy formulation 202, (ii) a non-fermented panax ginseng 40 with a non-fermented panax ginseng weight percent content from 2 weight percent to 15 weight percent based on the total pre-work out therapy formulation 202, (iii) a schizandra 50 with a schizandra weight percent content of 2 weight percent to 50 weight percent based on the total pre-work out therapy formulation 202, and (iv) a vitamin B₆ 60 with a vitamin B₆ content of from 2 weight percent to 15 weight percent based on the total pre-work therapy formulation 202. As with Formulations 1, 2 and 3, the epidermis temperature of an animal body with an epidermis varying from 90 degrees to 98 degrees Fahrenheit liquefies the dosage amount of the pre-work out therapy formulation 202 allowing transdermal absorption by the animal body for from 1 to 8 hours.

FIG. 4 shows three formulations, Formulation 4, Formulation 5, and Formulation 6 for the pre work out therapy formulation.

Formulation 4:

The Formulation 4 dosage amount of the pre-work out therapy formulation 202 has 2 weight percent of a guaranine extract as a caffeine 210 based on the total pre-work out therapy formulation 202, 2 weight percent of a non-fermented panax ginseng 40 based on the total pre-work out therapy formulation 202, 2 weight percent of a schizandra 50 based on the total pre-work out therapy formulation 202, and 2 weight percent of a vitamin B₆ 60 based on the total pre-work therapy formulation 202.

Formulation 4 also includes 5 weight percent of Ascorbic Acid 70, 5 weight percent of Folic Acid 80, 5 weight percent of Ginger Root Extract 90, 5 weight percent of Ginkgo Biloba Extract 100, and 5 weight percent of Lecithin 110.

In addition. Formulation 4 includes 8 weight percent of a white turmeric including tetrahydrocurcumin 240, 8 weight percent of a taurine 242, 8 weight percent of a niacinamide 244, and 8 weight percent of a methylcobalamine 246.

35 weight percent of a carrier 134 is used as well.

The ingredients are mixed in one pot then applied to the dosage amount pad.

Formulation 5:

Formulation 5 dosage amount includes 40 weight percent of a guaranine extract as a caffeine 210 based on the total pre-work out therapy formulation 202, 15 weight percent of a non-fermented panax ginseng 40 based on the total pre-work out therapy formulation 202, 30 weight percent of a schizandra 50 based on the total pre-work out therapy formulation 202, and 15 weight percent of a vitamin B₆ 60 based on the total pre-work therapy formulation 202.

Formulation 5 also includes 0 weight percent of Ascorbic Acid 70, 0 weight percent of Folic Acid 80, 0 weight percent of Ginger Root Extract 90, 0 weight percent of Ginkgo Biloba Extract 100, and 0 weight percent of Lecithin 110.

Additionally, Formulation 5 includes 0 weight percent of a white turmeric including tetrahydrocurcumin 240, 0 weight percent of a taurine 242, 0 weight percent of a niacinamide 244, and 0 weight percent of a methylcobalamine 246.

No carrier is used in this formulation.

The ingredients are mixed in one pot then applied to the dosage amount pad.

Formulation 6:

Formulation 6 only has 40 weight percent of a guaranine extract as a caffeine 210 based on the total pre-work out therapy formulation 202, 5 weight percent of a non-fermented panax ginseng 40 based on the total pre-work out therapy formulation 202, 50 weight percent of a schizandra 50 based on the total pre-work out therapy formulation 202, and 5 weight percent of a vitamin B₆ 60 based on the total pre-work therapy formulation 202.

The ingredients are mixed in one pot then applied to the dosage amount pad.

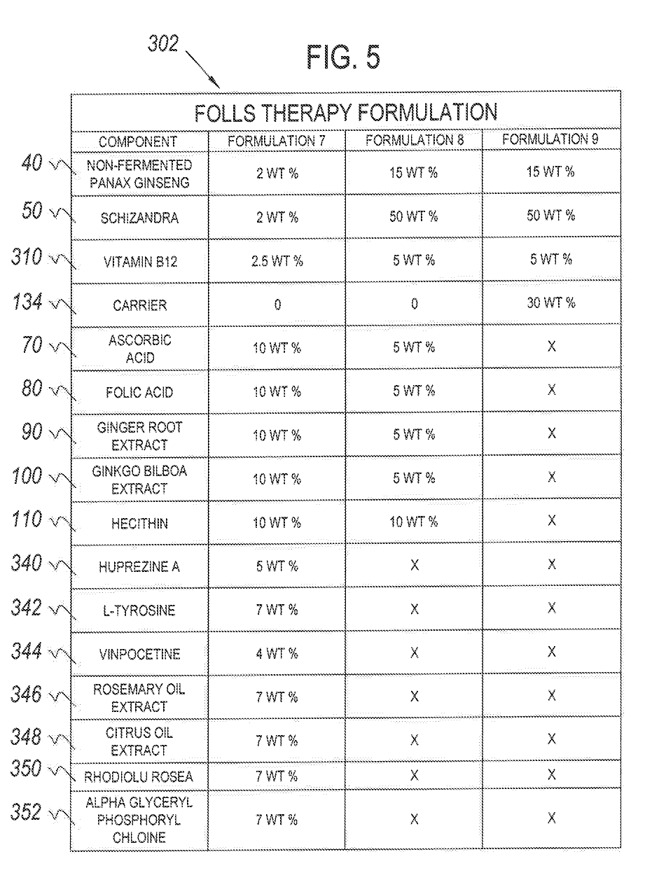

FIG. 5 shows a table of focus therapy formulations usable herein.

The dosage amount of the focus therapy formulation 302 includes (i) a non-fermented panax ginseng 40 having a non-fermented panax ginseng weight percent content from 2 weight percent to 15 weight percent based on the total focus therapy formulation 302, a schizandra 50 having a schizandra weight percent content of 2 weight percent to 50 weight percent based on the total focus therapy formulation 302, and a vitamin B₁₂ 310 having a vitamin B₁₂ content of from 2.5 weight percent to 5 weight percent based on the total focus therapy formulation 302; wherein the vitamin B₁₂ comprises methylcobalamin. The epidermis temperature of an animal body with an epidermis 25 varying from 90 degrees to 98 degrees Fahrenheit liquefies the dosage amount of the focus therapy formulation 302 allowing transdermal absorption by the animal body for from 1 to 8 hours.

FIG. 5 shows three formulations, Formulation 7, Formulation 8, and Formulation 9 for the pre work out therapy formulation.

Formulation 7:

Formulation 7 includes the dosage amount of the focus therapy formulation 302 with 2 weight percent of a non-fermented panax ginseng based on the total focus therapy formulation 302, 2 weight percent of a schizandra 50 based on the total focus therapy formulation 302, and 2.5 weight percent of a vitamin B₁₂ 310 based on the total focus therapy formulation 302; wherein the vitamin B₁₂ comprises methylcobalamin.

Additionally, Formulation 7 includes 10 weight percent of ascorbic acid 70, 10 weight percent of Folic Acid 80, 10 weight percent of Ginger Root Extract 90, 10 weight percent of Ginkgo Biloba Extract 100, and 10 weight percent of Lecithin 110.

Formulation 7 also includes 5 weight percent of Huprezine A 340, 7 weight percent of L-Tyrosine 342, 4 weight percent of vinpocetine 344, 7 weight percent of rosemary oil extract 346, 7 weight percent of citrus oil extract 348, 7 weight percent of Rhodiola Rosea 350, and 7 weight percent of Alpha glycerylphosphorylchloine 352.

No carrier is used in this formulation.

The ingredients are mixed in one pot then applied to the dosage amount pad.

Formulation 8:

Formulation 8 includes 15 weight percent of a non-fermented panax ginseng based on the total focus therapy formulation 302, 50 weight percent of a schizandra 50 based on the total focus therapy formulation 302, and 5 weight percent of a vitamin B₁₂ 310 based on the total focus therapy formulation 302; wherein the vitamin B₁₂ comprises methylcobalamin.

No other components are added to the formulation.

The ingredients are mixed in one pot then applied to the dosage amount pad.

Formulation 9:

Formulation 9 includes 15 weight percent of a non-fermented panax ginseng based on the total focus therapy formulation 302, 50 weight percent of a schizandra 50 based on the total focus therapy formulation 302, and 5 weight percent of a vitamin B₁₂ 310 based on the total focus therapy formulation 302; wherein the vitamin B₁₂ comprises methylcobalamin and 30 weight percent of a carrier.

No other components are added to the formulation.

The ingredients are mixed in one pot then applied to the dosage amount pad.

The following are three examples of the invention.

### Example One

Night Recovery Therapy Non-Ingestible Transdermal Sports Tape.

In this example, the night recovery therapy non-ingestible transdermal sports tape 10 has an occlusive nonwoven pre-cut tape 22. The tape can be rectangular 3.5 inches wide by 9.5 inches long.

The occlusive nonwoven pre-cut tape 22 is resistant to tearing and is flexible and elastic and made from an ethylene vinyl acetate sheet with a thickness of 0.02 to 0.031 ml.

A dosage amount pad 24 made from a cellulose material, such as woven cotton, is positioned on one side of the occlusive nonwoven pre-cut tape 22.

The dosage amount pad can support a dosage amount of a night recovery therapy formulation 26.

An adhesive seal material 28 made from a water resistant adhesive, is disposed on the occlusive nonwoven pre-cut tape 22 surrounding and covering the dosage amount pad 24.

The adhesive seal material can have a thickness from 0.2 mm to 0.4 mm.

The adhesive seal material can surround the dosage amount pad 24 covering a width that is at least ½ inch and can be up to 1 inch in width.

The adhesive seal material is configured to adhere the occlusive nonwoven pre-cut tape 22 to an animal body 28 with an epidermis 25, such as a foot of a human, providing a removable seal while preventing evaporation of water from epidermis 25.

The animal body 28 can be a human, a dog, a horse, or a rabbit.

A liner material 30 can be used, which can be an opaque vinyl material.

The liner material 30 can be flexible such as from 0.2 ml in thickness to 0.4 ml in thickness.

The liner material 30 covers the dosage amount pad 24 and the adhesive seal material 28.

The liner material 30 is configured to protect the dosage amount pad and adhesive seal material from drying until the animal body 28 with an epidermis 25 is ready to apply the night recovery therapy non-ingestible transdermal sports tape 10 to the animal body.

For this example, the dosage amount of the night recovery therapy formulation 26 has a non-fermented panax ginseng 40 having a non-fermented panax ginseng weight percent content of from 2 weight percent based on the total night recovery therapy formulation 26. Other amounts such as 8 weight percent can be used for other formulations.

This example includes a component known as schizandra 50 that has a schizandra weight percent content of 50 weight percent based on the total night recovery therapy formulation 26.

Also, this example includes a vitamin B₆ 60 of 2 weight percent based on the total night recovery therapy formulation 26.

The balance of the dosage formulation is a carrier 134 of 46 weight percent, such as polypropylene glycol to being the total amount of the night recovery therapy formulation to 100 weight percent or 20 weight percent of another ingredient.

The epidermis temperature of an animal body with an epidermis 25 can vary from 90 degrees to 98 degrees Fahrenheit liquefies the dosage amount of the night recovery therapy formulation 26 allowing transdermal absorption by the animal body for from 1 to 8 hours.

In embodiments of the night recovery therapy formulation, the carrier 134 weight percent can be reduced by 12 weight percent and the night recovery therapy non-ingestible transdermal sports tape can instead include 12 weight percent of Ascorbic Acid 70, Folic Acid 80, Ginger Root Extract 90, Ginkgo Biloba Extract 100, Lecithin 110 or combinations thereof.

In still other embodiments, the night recovery therapy formulation can have the carrier weight percent reduced by 36 weight percent and can instead include from 20 wt % to 36 weight percent of a lavender oil extract 120, a valerian root extract 122, a zizyphus seed extract 124, a He Shou Wu stem extract with Emodin 126, a Spirit Poria Sclerotium Extract 128, a Biota Seed Extract 130, a Gamma Aminobutyric Acid 132 or combinations thereof.

### Example Two

Pre-Work Out Non-Ingestible Transdermal Sports Tape

In this example, the pre-work out non-ingestible transdermal sport tape 200 can include an occlusive nonwoven pre-cut tape 22. The occlusive nonwoven pre-cut tape is resistant to tearing and is flexible and elastic.

In this example, the occlusive nonwoven pre-cut tape can be a square patch from 2.5 inches by 2.5 inches or circle from one inch to three inches.

The occlusive nonwoven pre-cut tape 22 can be made from an ethylene vinyl acetate sheet with a thickness of 0.01 ml to 0.40 ml.

A dosage amount pad 24 made from a woven synthetic polymer can be used. In embodiments, the dosage amount pad can be made from a woven organic cellulose can be positioned on one side of the occlusive nonwoven pre-cut tape.

The dosage amount pad supports a dosage amount of a pre-work out therapy formulation 202 that replaces in FIG. 1 the night recovery therapy formulation 26.

In this example, an adhesive seal material 28 made from a natural non-toxic water resistant adhesive can be disposed on the occlusive nonwoven pre-cut tape surrounding and covering the dosage amount pad 24.

Also, the adhesive seal material can surround the dosage amount pad with a width that is ¾ inch.

The adhesive seal material 28 is configured to adhere the occlusive nonwoven pre-cut tape to an animal body 28 with an epidermis 25, such as an arm, of a human or a gorilla or other primate, while simultaneously providing a removable seal while preventing evaporation of water from epidermis 26 of an animal body 28.

For this embodiment, the animal body can be a high school student that plays on the high school soccer team.

A liner material 30 can be used, such as brown kraft paper, or waxed paper, to cover the dosage amount pad 24 with the adhesive seal material 28.

The liner material 30 is configured to protect the dosage amount pad and adhesive seal material from drying until the animal body 28 with an epidermis 25 is ready to apply the pre-work out non-ingestible transdermal sports tape 200 to the animal body.

In this example, the dosage amount of the pre-work out therapy formulation 202 includes a guaranine extract as a caffeine 210 having a guanine extract weight percent content of 40 weight percent based on the total pre-work out therapy formulation 202.

The pre-work out therapy formulation of this example, has a non-fermented panax ginseng 40 weight percent content of 15 weight percent based on the total pre-work out therapy formulation 202.

The example can include a schizandra 50 having a schizandra weight percent content of 30 weight percent based on the total pre-work out therapy formulation 202.

Also, the example includes a vitamin B₆ 60 having a vitamin B₆ content of 15 weight percent based on the total pre-work therapy formulation 202.

The epidermis temperature of an animal body with an epidermis 25 varying from 90 degrees to 98 degrees Fahrenheit liquefies the dosage amount of the pre-work out therapy formulation 202 allowing transdermal absorption by the animal body 28 for from 1 to 8 hours.

In embodiments of the pre-work out formulation, one of the components can be reduced by 5 weight percent and the pre-work out formulation can include 5 weight percent of an ingredient selected from the group: Ascorbic Acid 70, Folic Acid 80, Ginger Root Extract 90, Ginkgo Biloba Extract 100, and Lecithin 110 or combinations thereof.

In still other embodiments, the pre-work therapy formulation can include up to 8 weight percent of an ingredient selected from the group: a white turmeric including tetrahydrocurcumin 240, taurine 242, niacinamide 244, methylcobalamine 246 and combinations thereof.

### Example Three

Focus Therapy Non-Ingestible Transdermal Sports Tape

In this example, the focus therapy non-ingestible transdermal ports tape 300 can include an occlusive nonwoven pre-cut tape 22.

The occlusive nonwoven pre-cut tape 22 is resistant to tearing and is flexible and elastic.

In embodiments, the occlusive nonwoven pre-cut tape 22 can be a triangular patch ranging in width at the widest section from 1.5 inches to 4 inches.

Additionally, the occlusive nonwoven pre-cut tape 22 can be made from a breathable polymer sheet with a thickness of 0.3 mm to 0.6 mm and which also can provide impact protection to the animal body.

A dosage amount pad 24 made from a nonwoven cellulose is positioned on one side of the nonwoven precut tape.

The dosage amount pad supports a dosage amount of a focus therapy formulation 302.

An adhesive seal material 28 made from a flexible polyester can disposed on the occlusive non-woven pre-cut tape 22 surrounding and covering the dosage amount pad 24.

The adhesive seal material can surround the dosage amount pad with a width that is ¼ inch.

The adhesive seal material is configured to adhere the occlusive non-woven pre-cut tape 22 to an animal body 28 with an epidermis 25 such as a shaved rabbit, providing a removable seal while preventing evaporation of water from epidermis of the animal body.

A liner material 30, which, in this example, can be a polyester, can be used to cover the dosage amount pad 24 and the adhesive seal material 28.

The liner material 30 is configured to protect the dosage amount pad and adhesive seal material from drying until the animal body with an epidermis is ready to receive the focus therapy non-ingestible transdermal sports tape 300 on the animal body.

The dosage amount of the focus therapy formulation 302 can include: (i) a non-fermented panax ginseng 40 having a non-fermented panax ginseng weight percent content of 15 weight percent based on the total focus therapy formulation 302, (ii) a schizandra 50 having a schizandra weight percent content of 50 weight percent based on the total focus therapy formulation 302, and (iii) a vitamin B₁₂ 310 having a vitamin B₁₂ content of 5 weight percent based on the total focus therapy formulation 302; and (iv) 30 weight percent of a carrier.

In this example, the vitamin B₁₂ can include methylcobalamin.

Also, the epidermis temperature of the animal body with an epidermis varies from 90 degrees to 98 degrees Fahrenheit and liquefies the dosage amount of the focus therapy formulation 302 allowing transdermal absorption by the animal body for from 1 to 8 hours.

In an embodiment, the focus therapy formulation can eliminate the carrier and replace the carrier with an ingredient selected from the group: ascorbic acid 70, Folic Acid 80, Ginger Root Extract 90, Ginkgo Biloba Extract 100, and Lecithin 110 and combinations thereof.

In embodiments, the focus therapy formulation can eliminate the carrier and replace the carrier with an ingredient selected from the group: Huprezine A 340, L-Tyrosine 342, vinpocetine 344, rosemary oil extract 346, citrus oil extract 348, Rhodiola Rosea 350, and Alpha glycerylphosphorylchloine 352.

In embodiments, each of the non-ingestible transdermal sports tape has as the occlusive nonwoven pre-cut tape and an ethylene vinyl acetate tape providing a resistant to tearing while providing flexing during adhesion to the animal body and simultaneously providing a flexible seal over the epidermis of the animal body while enabling removal of the non-ingestible transdermal sports tape without tearing the epidermis.

In embodiments, each of the non-ingestible transdermal sports tape can have the occlusive nonwoven pre-cut tape 22 configured with a transdermal reservoir 21 enabling an animal body to wear the dosage amount of the focus therapy formulation from 72 hours to 100 hours and receive a therapy dosage amount at a rate of 2 mg per hour for 125 hours

While these embodiments have been described with emphasis on the embodiments, it should be understood that within the scope of the appended claims, the embodiments might be practiced other than as specifically described herein.

What is claimed is:

1. A night recovery therapy non-ingestible transdermal sports tape comprising:
a. an occlusive nonwoven pre-cut tape, wherein the occlusive nonwoven pre-cut tape is resistant to tearing and is flexible and elastic;
b. a dosage amount pad positioned on one side of the occlusive nonwoven pre-cut tape, the dosage amount pad supporting a dosage amount of a night recovery therapy formulation;
c. an adhesive seal material disposed on the occlusive non-woven pre-cut tape surrounding and covering the dosage amount pad, the adhesive seal material configured to adhere the occlusive non-woven pre-cut tape to an animal body 28 with an epidermis providing a removable seal while preventing evaporation of water from epidermis of the animal body; and
d. a liner material covering the dosage amount pad with the adhesive seal material, the liner material configured to protect the dosage amount pad and adhesive seal material from drying until the animal body is ready to apply the night recovery therapy non-ingestible transdermal sports tape to the animal body; and
wherein the dosage amount of the night recovery therapy formulation comprises:
a non-fermented panax ginseng having a non-fermented panax ginseng weight percent content from 2 weight percent to 15 weight percent based on the total night recovery therapy formulation;
i. a schizandra having a schizandra weight percent content from 2 weight percent to 50 weight percent based on the total night recovery therapy formulation; and
ii. a vitamin B₆ having a vitamin B₆ weight percent content from 2 weight percent to 15 weight percent based on the total night recovery therapy formulation; and
wherein the epidermis temperature of an animal body with an epidermis varying from 90 degrees to 98 degrees Fahrenheit liquefies the dosage amount of the night recovery therapy formulation allowing transdermal absorption by the animal body for from 1 to 8 hours.

2. The night recovery therapy non-ingestible transdermal sports tape of claim 1, wherein the occlusive nonwoven pre-cut tape is an ethylene vinyl acetate tape providing a resistant to tearing while providing flexing during adhesion to the animal body and simultaneously providing a flexible seal over the epidermis of the animal body while enabling removal of the night recovery therapy non-ingestible transdermal sports tape without tearing the epidermis.

3. The night recovery therapy non-ingestible transdermal sports tape of claim 1, comprising 20 weight percent of an ingredient selected from the group: Ascorbic Acid, Folic Acid, Ginger Root Extract, Ginkgo Biloba Extract, and Lecithin.

4. The night recovery therapy non-ingestible transdermal sports tape of claim 1, comprising 20 weight percent of an ingredient selected from the group: lavender oil extract, valerian root extract, zizyphus seed extract, He Shou Wu stem extract with Emodin, Spirit Poria Sclerotium Extract, Biota Seed Extract, and Gamma Aminobutyric Acid.

5. The night recovery therapy non-ingestible transdermal sports tape of claim 1, wherein the occlusive nonwoven pre-cut tape is configured with a transdermal reservoir enabling an animal body to wear the dosage amount of the night recovery formulation from 72 hours to 100 hours and receive a therapy dosage amount at a rate of 2 mg per hour for 125 hours

6. A pre-work out non-ingestible transdermal sports tape comprising:
a. an occlusive nonwoven pre-cut tape, wherein the occlusive nonwoven pre-cut tape is resistant to tearing and is flexible and elastic;
b. a dosage amount pad positioned on one side of the occlusive nonwoven pre-cut tape, the dosage amount pad supporting a dosage amount of a pre-work out therapy formulation;
c. an adhesive seal material disposed on the occlusive nonwoven pre-cut tape surrounding and covering the dosage amount pad, the adhesive seal material configured to adhere the occlusive nonwoven pre-cut tape to an animal body with an epidermis providing a removable seal while preventing evaporation of water from epidermis of the animal body; and
d. a liner material covering the dosage amount pad with the adhesive seal material, the liner material configured to protect the dosage amount pad and adhesive seal material from drying until the animal body with an epidermis is ready to apply the pre-work out non-ingestible transdermal sports tape to the animal body; and
wherein the dosage amount of the pre-work out therapy formulation comprises:
a guaranine extract as a caffeine having a guanine extract weight percent content from 2 weight percent to 40 weight percent based on the total pre-work out therapy formulation;
a non-fermented panax ginseng having a non-fermented panax ginseng weight percent content from 2 weight percent to 15 weight percent based on the total pre-work out therapy formulation;
a schizandra having a schizandra weight percent content of 2 weight percent to 50 weight percent based on the total pre-work out therapy formulation;
a vitamin B₆ having a vitamin B₆ content of from 2 weight percent to 15 weight percent based on the total pre-work therapy formulation;
wherein the epidermis temperature of an animal body with an epidermis varying from 90 degrees to 98 degrees Fahrenheit liquefies the dosage amount of the pre-work out therapy formulation allowing transdermal absorption by the animal body for from 1 to 8 hours.

7. The pre-work out non-ingestible transdermal sports tape of claim 6, wherein the occlusive nonwoven pre-cut tape is an ethylene vinyl acetate tape providing a resistant to tearing while providing flexing during adhesion to the animal body and simultaneously providing a flexible seal over the epidermis of the animal body while enabling removal of the pre-work out non-ingestible transdermal sports tape without tearing the epidermis.

8. The pre-work out non-ingestible transdermal sports tape of claim 6, comprising 5 weight percent of an ingredient selected from the group: Ascorbic Acid, Folic Acid, Ginger Root Extract, Ginkgo Biloba Extract, and Lecithin.

9. The pre-work out non-ingestible transdermal sports tape of claim 6, comprising 8 weight percent of an ingredient selected from the group: a white turmeric including tetrahydrocurcumin, taurine, niacinamide, and methylcobalamine.

10. The pre-work out therapy non-ingestible transdermal sports tape of claim 6, wherein the occlusive nonwoven pre-cut tape is configured with a transdermal reservoir enabling an animal body to wear the dosage amount of the pre-work out therapy formulation from 72 hours to 100 hours and receive a therapy dosage amount at a rate of 2 mg per hour for 125 hours.

11. A focus therapy non-ingestible transdermal sports tape comprising:
a. an occlusive nonwoven pre-cut tape, wherein the occlusive nonwoven pre-cut tape is resistant to tearing and is flexible and elastic;
b. a dosage amount pad positioned on one side of the occlusive nonwoven pre-cut tape, the dosage amount pad supporting a dosage amount of a focus therapy formulation;
c. an adhesive seal material disposed on the occlusive nonwoven pre-cut tape surrounding and covering the dosage amount pad, the adhesive seal material configured to adhere the occlusive nonwoven pre-cut tape to an animal body with an epidermis providing a removable seal while preventing evaporation of water from epidermis of the animal body; and
d. a liner material covering the dosage amount pad with the adhesive seal material, the liner material configured to protect the dosage amount pad and adhesive seal material from drying until the animal body with an epidermis is ready to apply the focus therapy non-ingestible transdermal sports tape to the animal body; and
wherein the dosage amount of the focus therapy formulation comprising:
i. a non-fermented panax ginseng having a non-fermented panax ginseng weight percent content from 2 weight percent to 15 weight percent based on the total focus therapy formulation;
ii. a schizandra having a schizandra weight percent content of 2 weight percent to 50 weight percent based on the total focus therapy formulation.
iii. a vitamin B₁₂ having a vitamin B₁₂ content of from 2.5 weight percent to 5 weight percent based on the total focus therapy formulation; wherein the vitamin B₁₂ comprises methylcobalamin; and
wherein the epidermis temperature of an animal body with an epidermis 25 varying from 90 degrees to 98 degrees Fahrenheit liquefies the dosage amount of the focus therapy formulation allowing transdermal absorption by the animal body for from 1 to 8 hours.

12. The focus therapy non-ingestible transdermal sports tape of claim 11, wherein the occlusive nonwoven pre-cut tape is an ethylene vinyl acetate tape providing a resistant to tearing while providing flexing during adhesion to the animal body and simultaneously providing a flexible seal over the epidermis of the animal body while enabling removal of the focus therapy non-ingestible transdermal sports tape without tearing the epidermis.

13. The focus therapy non-ingestible transdermal sports tape of claim 11, comprising from 5 weight percent to 10 weight percent of an ingredient selected from the group: ascorbic acid, Folic Acid, Ginger Root Extract, Ginkgo Biloba Extract, and Lecithin.

14. The focus therapy non-ingestible transdermal sports tape of claim 11, comprising from 4 to 7 weight percent of an ingredient selected from the group: Huprezine A, L-Tyrosine, vinpocetine, rosemary oil extract, citrus oil extract, Rhodiola Rosea, and Alpha glycerylphosphorylchloine.

15. The focus therapy non-ingestible transdermal sports tape of claim 11, wherein the occlusive nonwoven pre-cut tape is configured with a transdermal reservoir enabling an animal body to wear the dosage amount of the focus therapy formulation from 72 hours to 100 hours and receive a therapy dosage amount at a rate of 2 mg per hour for 125 hours.
